(19) United States
(12) Patent Application Publication
(10) Pub. No.: US 2024/0035806 A1
(43) Pub. Date: Feb. 1, 2024

(54) Line-field swept source OCT system for monitoring biological systems

(71) Applicant: KineoLabs, Billerica, MA (US)

(72) Inventor: Walid A. Atia, Jamaica Plain, MA (US)

(21) Appl. No.: 18/359,094

(22) Filed: Jul. 26, 2023

Related U.S. Application Data
(60) Provisional application No. 63/393,628, filed on Jul. 29, 2022.

(51) Int. Cl. G01B 9/02091; G01B 9/02004; G01B 9/02055; G06T 11/00

(52) U.S. Cl. CPC G01B 9/02091; G01B 9/02004; G01B 9/02077; G06T 11/005

(57) ABSTRACT

A compact possibly all free-space line-field swept source OCT system with a tunable cat's-eye laser is used to control and optimize the operation of a biological system.

### RELATED APPLICATIONS

This application claims the benefit under 35 USC 119(e) of U.S. Provisional Application No. 63/393,628, filed on Jul. 29, 2022, which is incorporated herein by reference in its entirety.

### BACKGROUND OF THE INVENTION

Optical coherence tomography (OCT) is a cross-sectional, non-invasive imaging modality that is used in many areas of medical imaging. For example, in ophthalmology, OCT has been widely used for imaging the retina, choroid and anterior segment. Functional imaging of the blood velocity and vessel microvasculature is also possible.

Fourier-domain OCT (FD-OCT) has recently attracted more attention because of its high sensitivity and imaging speed compared to time-domain OCT (TD-OCT), which uses an optical delay line for mechanical depth scanning with a relatively slow imaging speed. The spectral information discrimination in FD-OCT is accomplished either by using a dispersive spectrometer in the detection arm (spectral domain or SD-OCT) or rapidly scanning a swept laser source (swept-source OCT or SS-OCT).

Compared to spectrometer-based SD-OCT, SS-OCT has several advantages, including its robustness to motion artifacts and fringe washout, lower sensitivity roll-off and higher detection efficiency.

Many different approaches have been implemented to develop high-speed swept sources for SS-OCT. One approach employs a semiconductor optical amplifier (SOA) based ring laser design (see for example Yun et al “High-speed optical frequency-domain imaging” Opt. Express 11:2953 2003 and Huber et al “Buffered Fourier domain mode locking: unidirectional swept laser sources for optical coherence tomography imaging at 370,000 lines/s,” Opt. Express 13, 3513 2005). Short cavity lasers (see for example Kuznetsov et al “Compact Ultrafast Reflective Fabry-Perot Tunable Lasers For OCT Imaging Applications,” Proc. SPIE 7554:75541F 2010) are another example. SOA based ring laser designs have been practically limited to positive wavelength sweeps (increasing wavelength) because of the significant power loss that occurred in negative tuning. This has been attributed to four-wave mixing (FWM) in SOAs causing a negative frequency shift in intracavity light as it propagates through the SOA (Bilenca et al “Numerical study of wavelength-swept semiconductor ring lasers: the role of refractive-index nonlinearities in semiconductor optical amplifiers and implications for biomedical imaging applications,” Opt. Lett. 31: 760-762 2006).

A commercially available short cavity laser (Axsun Technologies Billerica, MA) in excess of 100 kHz has been reported (see for example Kuznetsov et al “Compact Ultrafast Reflective Fabry-Perot Tunable Lasers for OCT Imaging Applications,” Proc. SPIE 7554: 75541F 2010). Short cavity lasers enable a significant increase in sweep speeds over conventional swept laser technology because the time needed to build up lasing from spontaneous emission noise to saturate the gain medium is greatly shortened (R. Huber et al “Buffered Fourier domain mode locking: unidirectional swept laser sources for optical coherence tomography imaging at 370,000 lines/s,” Opt. Express 13:3513 2005). However, the effective duty cycle of the bidirectional sweeping short cavity laser was limited to less than 50% because of the FWM effects mentioned above. The effective repetition rate of the laser is thus limited.

More recently, tunable vertical cavity surface emitting lasers (VCSELs) have been offered by Thorlabs and Axsun Technologies. The short cavities implicit in this technology enables even higher speed sweeping.

Other methods have also been proposed to increase the effective repetition rates of SS-OCT systems including sweep buffering with a delay line, and multiplexing of multiple sources, thereby increasing the duty cycle of the laser. The method used to multiplex these sweeps together may include components that introduce orthogonal polarizations to the sweeps originating from different optical paths. Combining diverse polarizations at a polarization beamsplitter is a very light efficient way of transmitting the light to a single beam path.

Goldberg et al. demonstrated a ping-pong laser configuration for high-speed SS-OCT system that achieves a doubling of the effective A-line rate by interleaving sweeps of orthogonal polarization in the same cavity (see Goldberg et al “200 kHz A-line rate swept-source optical coherence tomography with a novel laser configuration” Proceedings of SPIE v.7889 paper 55 2011).

Potsaid et al. demonstrated another method to double the effective repetition rate of a swept source laser by buffering and multiplexing the sweep of a single laser source (see Potsaid et al “Ultrahigh speed 1050 nm swept source/Fourier domain OCT retinal and anterior segment imaging at 100,000 to 400,000 axial scans per second” Opt. Express 18: 20029-20048 2010). However, the long fiber spool will cause a significant birefringence to the laser output.

Other architectures exist for SS-OCT that reduce the performance requirements for the swept laser source, however. Fechtig, et al. in an article entitled Line-Field parallel swept source MHz OCT for structural and functional retinal imaging, Biomedical Optics Express 716, vol. 6, no. 3, (2015) describes a system that achieves 1 MHz equivalent A-scan rates by combining a lower sweep rate laser with a linear or line-scan sensor.

At the same time, many applications in biology, medicine, pharmaceutical research and other areas use techniques in which genetic materials are introduced into cells. The term “transformation” is often used when working with bacteria or non-animal eukaryotic cells, including plant cells. “Transfection” almost always refers to work on eukaryotic cells, while “transduction” typically applies to virus-mediated gene transfer into eukaryotic cells.

Materials of interest can include not only DNA, siRNA, mRNA, RNP complexes, but also small molecules or proteins such as antibodies. In many cases, the transfer of such a “cargo” material involves opening transient pores or “holes” in the cell membrane to allow its uptake and thus alter or genetically modify the cells.

One common technique used to temporarily permeabilize cells is electroporation. Parameters considered when developing electroporation procedures include cell properties (cell size, shape, membrane structure, surface charge, for example), the cell environment, and attributes of the applied electric field, (e.g., pulse intensity, number of pulses, pulse duration, pulse shape and/or frequency). It is generally believed that membrane permeabilization during electroporation occurs after the applied electric field induces a threshold value in the transmembrane potential or “electroporation threshold” and that, at a given applied electric field, there is a threshold for the number of pulses and pulse length, needed for successful electroporation. The Schwan equation and related derivations are often used to estimate a cell's transmembrane potential that develops in response to relevant experimental parameters including applied field, cell size, conductivities of media, cellular cytosol, and cell membrane, and membrane thickness (“Analytical Description of Transmembrane Voltage Induced by Electric Fields on Spheroidal Cells”, Biophysical Journal, Volume 79 August 2000 670-679).

Traditionally, the genetic modification of cells by electroporation has been conducted as a bulk, batch process using cuvettes.

As cellular therapies move toward large-scale production using allogenic rather than autologous sources of cells, bioprocessing of cellular therapies using bulk methods are increasingly becoming intractable and replaced with continuous systems.

For continuous systems, a microfluidic hydrodynamic sheath flow configuration is often used. Such systems have microfluidic channel arrangements for pushing cells from side streams containing, for example, a cell culture medium, to a central stream containing an electroporation buffer. Electroporation can be conducted in an assembly in which two or more microfluidic channels are provided in a parallel configuration and in which various layers can be stacked together to form a laminate type structure. Each microfluidic channel is provided with a pair of electrodes, preferably constructed to withstand long-lasting, continuous and high throughput operations.

### SUMMARY OF THE INVENTION

The present invention concerns a potentially compact line-field SS-OCT system. It can be an entirely free-space system in that no optical fiber is required. Thus, the system can also be very compact and completely integrated on a single bench. This is used to monitor cells in microfluid devices and track the movement of the cells such as through acoustic focusing and defocusing for electroporation, for example.

In general, according to one aspect, the invention features a biological system comprising: devices for modifying cells and a line-field swept source OCT system for monitoring the cells.

In embodiments, a controller is provides that uses information from the line-field swept source OCT to control the devices.

In further aspects of embodiments, the devices include pumps feedback controlled by the controller. The devices can further include at least one incubator and a line-field swept source OCT system for monitoring cells in the incubator. Also, the devices can further include at least one buffer exchange device and a line-field swept source OCT system for monitoring cells in the buffer exchange device. In addition, the devices can further include flow electroporation device and a line-field swept source OCT system for monitoring cells in the flow electroporation device.

In a preferred embodiment, the wherein the line-field swept source OCT system includes a base, a swept laser on the base, a beamsplitter on the base for dividing the beam from the swept laser between a reference arm and a sample arm, and a line-field sensor for detecting light from the reference arm and the sample arm.

In general, according to one aspect, the invention features a control method for a biological system comprising modifying cells in the biological system, monitoring the cells in the biological system with a line-field swept source OCT system and controlling the biological system based on information from the line-field swept source OCT system.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is perspective views of a line field swept source optical coherence tomography system for microfluidic monitoring according to the present invention.

FIG. 2 is a schematic diagram showing an apparatus for a hands-free, continuous flow transfection of cells under the monitoring of the OCT system 100.

FIG. 3 is a top view showing a device component configured to support a flow arrangement that uses a central stream and side streams.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

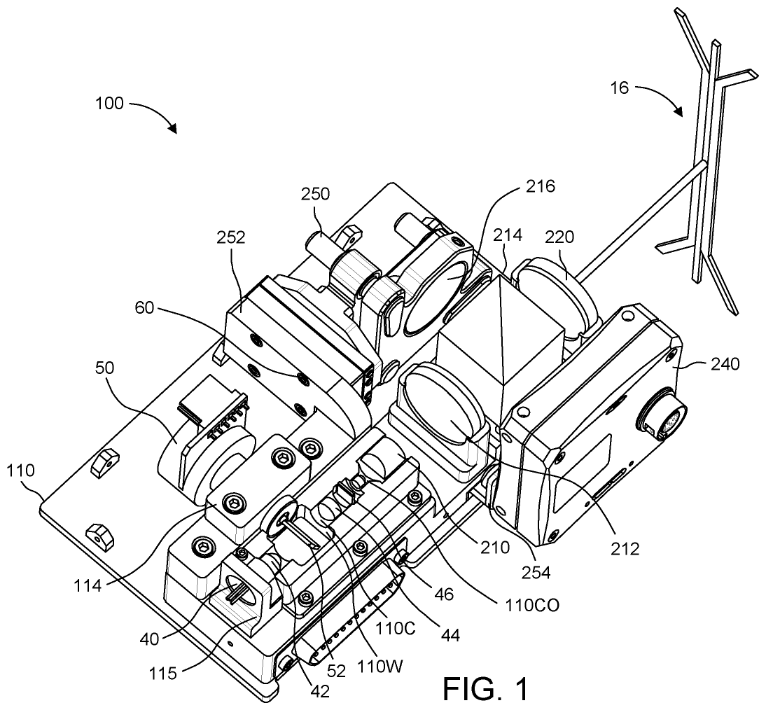

FIG. 1 shows a line-field free-space swept-source optical coherence tomography system (SS-OCT) 100, which has been constructed according to the principles of the present invention.

The system's swept source laser employs a cats-eye architecture. The laser's amplification is provided by a GaAlAs gain chip in one example. The gain chip amplifies light in the wavelength range of about 800 to 900 nanometers which is an effective water window. Preferably, the gain chip is an edge emitting, single angled facet device. Preferably its center wavelength is around 840 nanometers, which is useful for applications such as ophthalmic imaging but also flowing cells in microfluidic devices. Another advantage of this wavelength range is that it can be detected with silicon, e.g., CMOS or CCD, imagers.

Other material systems can be selected for the gain chip, however. Common material systems are based on III-V semiconductor materials, including binary materials, such as GaN, GaAs, InP, GaSb, InAs, as well as ternary, quaternary, and pentenary alloys, such as InGaN, InAlGaN, InGaP, AlGaAs, InGaAs, GaInNAs, GaInNAsSb, AlInGaAs, InGaAsP, AlGaAsSb, AlGaInAsSb, AlAsSb, InGaSb, InAsSb, and InGaAsSb. Collectively, these material systems support operating wavelengths from about 400 nanometers (nm) to 2500 nm, including longer wavelength ranges extending into multiple micrometer wavelengths. Semiconductor quantum well, quantum cascade and quantum dot gain regions are typically used to obtain especially wide gain and spectral emission bandwidths, and support operation up to 250 um in wavelength. Quantum well layers may be purposely strained or unstrained depending on the exact materials and the desired wavelength coverage.

In the preferred embodiment, the gain chip is mounted in a TO-can type hermetic package 40. This protects the chip from dust and the ambient environment including moisture. In some examples, the TO-can package has an integrated or a separate thermoelectric cooler. In a current embodiment, the gain chip is run coolerless with no thermoelectric cooler so that the chip heat is dissipated through bracket 115 and into a base 110.

The free space beam from the package is diverging in both axes (x, y). It is collimated by a collimating lens 42. The resulting collimated beam is received by a cat's eye focusing lens 44, which focuses the light onto a cat's eye mirror/output coupler 46. This defines the other end of the laser cavity, extending between the mirror/output coupler and the back/reflective facet of the gain chip in the TO-can 40 and forms a cats-eye laser cavity.

The collimated light between the collimating lens and the cat's eye focusing lens passes through a bandpass filter 52. This is preferably a thin film interference filter that provides a pass band of approximately 0.3 nanometers (nm). More generally, it is usually between 0.1 and 2 nanometers. Even more generally, it is between 0.05 nm to 5 nm filter linewidth. Note that the linewidths are measured at full width, half max.

The bandpass filter 52 is held on an arm of a galvanometer 50. This allows for tilting of the bandpass filter in the collimated beam to thereby tilt tune the filter and thus change the passband to scan or sweep the wavelength of the swept laser.

Tuning speed specifications for galvanometer generally range from 0.1 Hz to 50 kHz. For the higher speeds, a 25 kHz resonant galvo can be used with bi-directional tuning, but higher and lower speeds can be used. Wavelength tuning speed is usually given in nm/sec, so for a typical 100 Hz tuning where a line-speed camera at 100 kHz will give 1000 sampled bandwidth points and 70 nm tuning range, this would give 70 nm/10 msec=7000 nm/sec. In general, the tuning speed should be between 3000 nm/sec 70000 nm/sec.

Tuning range specifications: For industrial and biological imaging with low-cost CMOS cameras, 840 nm center wavelength is an ideal water window, and a minimum of 30 nm tuning range with 70 nm tuning for good resolution of <8 micrometers in air. In general, it should be between 30 nm and 100 nm.

The size of the collimated beam is important for many applications. As a general rule, a smaller beam results in higher divergence resulting in a larger cone half angle (CHA). This reduces the minimum line width over angle for a tunable filter. In the current embodiment, the collimated beam is not less than 1 millimeter (mm) and is preferably greater than 2 mm for retinal OCTG application. It can be smaller, however, for many spectroscopy applications in the infrared, visible or ultraviolet. In general, the CHA should be less than 0.04×.02 degrees and preferably about 0.02×.01 degrees or less.

The light from the gain chip is polarized. In the common architectures, the polarization is horizontal or parallel to the epitaxial layers of the edge-emitting gain chip. In the preferred configuration, the filter is oriented to receive the S polarization in order to maintain narrow line width of the filter as it is tilt tuned. On the other hand, the P polarization broadens drastically at large tilt angles. S polarization has higher loss at larger tilt angles than P. So, the filter design needs to address these issues by providing a low enough loss across the tuning band for S.

In general, the present cat's-eye laser configuration provides a number of advantages. It provides low loss, low tolerance, repeatable stable operation since lower angle wavelength change over grating-based lasers.

The mirror/output coupler 46 will typically reflect about 80% of the light back into the laser's cavity and transmits about 20% of light. Often, the transmitted light is collimated with the help of an output lens. More generally, the mirror/output coupler can reflect from 10% to 99% of light (transmitting 90% to 1%, respectively), depending on the output power and laser cavity loss desired. Higher reflectivity results in lower loss cavities and thus wider laser tuning range where gain exceeds loss, but results in lower output power.

In some embodiments, an iris or mask is added typically after the output coupler to clip the beam edge. This reduces power fluctuations as the beam wanders due to refraction in the tilting bandpass filter.

In the illustrated example, the fast axis of the chip is oriented horizontally in the figure, with the epitaxial layers of the chip being oriented vertically. Thus the beam transmitted through the mirror/output coupler 46 is elliptical with the long axis of the beam being vertical. This diverging elliptical beam is collimated by collimating output lens 60. Generally, the elliptical beam is between 1-4 millimeter wide in the long axis and about 0.5-2 millimeters in the short axis.

The elliptical beam is received by a line forming lens 210 such as a Powell lens, in one example. This generates a beam that has a less Gaussian and more of a flat-top power distribution in along the narrow axis which is much preferred to a Gaussian distribution as it gives a uniform signal to noise ratio (SNR) over the image and does not have a large hot spot, allowing for a higher safe optical power of the beam and further improving the SNR.

As shown, the diverging light along the fast diverging beam from the fanned out rays of the Powell lens is collimated by a final lens 212 while the collimated part of the beam on the opposite axis is focused. This creates an extended beam on one axis and a collimated beam on the other axis that then produces a focused line on the retina.

The system is supported on a base 110. The TO-can 40 is held in an L-shaped mount 115 that holds the TO-can 40 above the base.

The base 110 also has a mirror well 110W.

For holding the various components, it has a series of cradles or V-groove optical element mounting locations formed into the top surface of the frame. These include cats-eye focusing lens v-groove cradle 110C, collimating output lens cradle 110CO, a cats-eye collimating lens v-groove cradle 110F.

Finally, galvanometer clamp 114 secures the galvanometer 50 to the base 110.

Light from the laser and specifically the Powell lens fanned out rays is collimated by an interferometer collimating lens 212.

The light from the interferometer from the fanned out Powell lens rays to the collimating lens 212 passes in free space to a beam splitter 214 of the OCT interferometer. The beamsplitter 214 divides the light between the reference arm defined by a reference arm mirror 216 and the sample arm that ends with a sample 16, such as microfluid device.

The light from the sample is collected by a collection and collimating lens 220 and the light from the two arms returns to the beamsplitter 214 to be combined to form light interference in a line-field sensor 240.

Light enters the line-field sensor 240 through its aperture to be received by the sensor chip, which is preferably CMOS or CCD device. These are silicon devices that work at the 800-900 nm wavelength. One commercially available camera is the NECTA series sold by Alkeria Srl. These CMOS-sensor devices have a USB-3 interface having at least 1024 and preferably 2048 or more pixels. The pixel sizes range from about over 2 micrometers to as large as 10 micrometers.

The output from the sensor is readout by a processor. The results can be stored in the processor and/or displayed on display. The Fourier transform of the interference light reveals the profile of scattering intensities at different path lengths, and therefore scattering as a function of depth (z-direction) in the sample (see for example Leitgeb et al, “Ultrahigh resolution Fourier domain optical coherence tomography,” Optics Express 12(10):2156 2004). The profile of scattering as a function of depth is called an axial scan (A-scan). A set of A-scans measured at neighboring locations in the sample produces a cross-sectional image (tomogram or B-scan) of the sample. A collection of B-scans makes up a data cube or cube scan.

Typically an additional galvanometer driven scanning mirror is provided between the beamsplitter 214 and the sample, so that the beam of light is scanned in one axis.

In terms of packaging, the reference arm mirror 216 is held in a mirror bracket 250 on a translation stage 252 for pathlength adjustment. The translation stage 252 is mounted to the base 110 and specifically an arm of the base.

The beamsplitter 214 is also mounted to the base 110. The line-field sensor 240 is mounted to a camera bracket 254 that is mounted to the base 110.

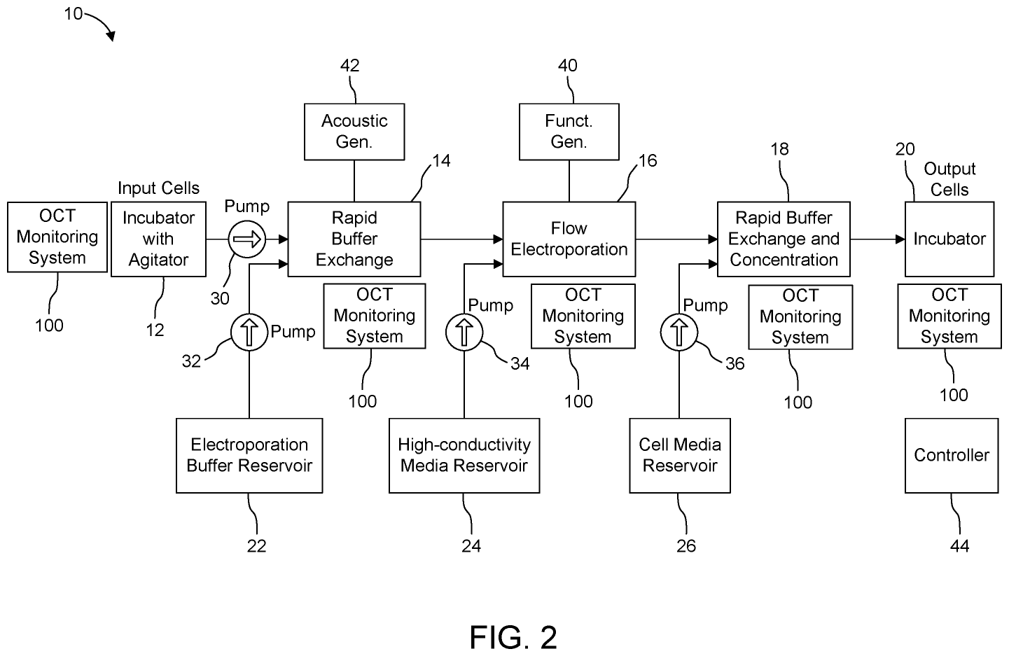

Shown in FIG. 2, for example, is biological system 10 which includes several components or modules: incubator 12, (first) buffer exchanger 14, electroporation assembly 16, (second) buffer exchanger 18 and (second) incubator 20. The movement of cells in such a system is monitored using the line field swept source optical coherence tomography system 100 of FIG. 1.

The biological system can further include one or more reservoirs such as reservoirs 22, 24 and 26, for example and pumps 30, 32, 34 and 36. Voltages required for electroporation are provided by electrical function generator 40 and acoustic energy is provided via acoustic function generator 42. The system is controlled by controller 44. Often the controller is a microprocessor in a computer system such as a single board computer system. In other cases, the controller is a microcontroller with integrated memory and analog to digital converters and digital to analog converters.

Either or both incubators 12 and 20 (disposed, respectively, upstream of and downstream of electroporation assembly 16) can be benchtop incubation chambers configured for housing cells before and/or after electroporation and can have an internal volume of about 0.3 to about 50 liters (L). In many cases one or both incubators have a miniaturized design.

An OCT system 100 as described in FIG. 1 is used to monitor the cells and their movement in the input incubator 12 and another OCT system 100 as described in FIG. 1 is used to monitor the cells and their movement in the output incubator 20. Each of these OCT systems provides cell viability and cell location information to the controller 44 which uses the information to control the biological system 10.

Typically, incubator 12 and/or 20 is/are provided with means for transferring cells to and/or from the incubators.

For example, incubator 12 can be equipped with a receptacle for a cell container or reservoir (a flask, conical tube, etc.). The container can be a sealed, sterile container such as a blood bag, for instance. In illustrative examples, it provides cells at a concentration of 10⁵ to 5×10⁸ cell/mL) suspended in a high-conductivity (e.g., about 1 to 2 S/m) culture buffer, such as TexMACS or RPMI (Roswell Park Memorial Institute) medium for T cells.

A closed loop or another suitable arrangement can be included to control the cell culture conditions, e.g., the incubator temperature (often maintained at 37° C.), the incubator gas composition (CO₂ and/or humidity levels, for instance), sensors for metabolic or general processing readouts (pH, 02, etc.) and so forth. This control is based on the monitoring of the incubator OCT system 100 which is able to assess the health and density of the cells. This information is passed to the controller 44 which then provides operates the incubator to optimize cell health.

Examples of cells that can be housed in incubator 12 include suspension cells such as primary T cells, NK cells, hematopoietic stem cells, or adherent cells such as MSCs, CHO cells, and many others. In some implementations, incubators 12 and/or 20 are designed or adapted to support membrane-bound structures such as liposomes, exosomes, micelles and so forth. Suitable buffers and conditions for keeping these targets stable can be selected as known in the art and/or determined experimentally. The OCT systems 100 are also used to monitor the distribution on these membrane bound structures.

With cells (or other membrane-bound structures) that may not be neutrally buoyant in the culture medium, the incubator can be fitted with an agitation mechanism for generating a (gentle) movement in the container that houses the cells, reducing, minimizing or preventing settling or sinking. This helps to ensure that the concentration of cells that is delivered into downstream operations (media exchange and electroporation) is controlled and consistent.

In some cases, rather than utilizing separate incubator chambers for the start and end of the process (see, e.g., elements 12 and 20 in FIG. 2), the entire system is housed in an incubator chamber.

In many of the embodiments described herein, electroporation assembly 16 supports electroporation processes conducted in continuous fashion, using a sheath flow configuration, in a microfluidic channel, for example. One specific implementation brings cells and cargo into contact in a central flow or stream, that typically utilizes a low conductivity fluid, also referred to herein as an electroporation fluid. The central stream flows between two sheath (also referred to as “side” or “lateral”) streams that typically employ high conductivity fluids. The difference in conductivity between the center and side fluids leads to a concentration of the electric field (supplied by voltage generator 40) in the central (low-conductivity) region of the flow, allowing an effective amplification of the electric field strength and preventing cells in the central stream from coming into physical contact with the electrodes.

Considering that preferred media for cell cultures typically have high electrical conductivity and the sheath flow arrangement described above preferably places the cells in a low conductivity medium during electroporation, system 10 uses buffer exchange arrangement 14 for transferring cells from the cell culture medium to an electroporation buffer medium. In many embodiments, the rapid buffer exchange involves driving cells from one flow stream into another acoustically, with acoustic frequencies being generated through component 42.

Preferably, the efficiency of the buffer exchange device is monitored by an OCT system 100 that is integrated into the buffer exchange arrangement 14 to monitor and provide feedback control. The feedback includes the flow rate of cells, the location of cells in the device. This information is provided to the controller enabling feedback control of the buffer exchange device 14.

After electroporation, cells can be transferred from the electroporation medium into a cell medium. Another buffer exchange module 18 further includes a cell concentration function. Traditionally, cell concentration is typically accomplished in batch processes using centrifugation, but could be accomplished in flow configurations using acoustophoresis, dielectrophoresis, electrophoresis, inertial effects, or integrated porous membranes or sieves.

In one embodiment, at least one of buffer exchange devices 14 and 18 is a rapid buffer exchange device and cell concentrator. Another embodiment utilizes a design in which one or both buffer exchange (switching) modules and the electroporation device 16 (which can employ the sheath flow configuration described above) are integrated into a single apparatus. In a further embodiment, at least one buffer exchange is conducted in a device that is separate from the electroporation device. In one example of this approach, buffer exchanger 14, flow electroporation assembly 16 and buffer exchanger 18 are connected to one another by conduits, e.g., suitable tubing, that can provide fluid communication between these components.

Some aspects employ acoustically-driven rapid buffer switching in both devices 14 and 18. In other aspects, non-acoustically-driven buffer switching is utilized in at least one of the buffer exchange devices. One implementation utilizes an acoustically-driven buffer exchange device 14 and a non-acoustically-driven buffer exchange device 18.

Output cells are collected in incubator 20. In one example, these cells are primary human T cells that contain mRNA. Such cells can be used in gene editing applications, or as transient therapeutic systems (mRNA CAR-T). In other examples, the output cells are used for protein or extracellular vesicle production (e.g., modified CHO cells or MSCs).

Throughout the system, flow is driven (actuated) by a pump system, including pumps 30, 32, 34, 36 that are commercial, off-the shelf pumps often of peristaltic design. Other suitable pump types can be employed. In general, one pump is used to actuate flow of the cell suspension out of the first incubation chamber and through the entire system with a flow rate that ranges, e.g., from 100 μL/min to 2 mL/min. One additional pump is needed for each buffer exchange that occurs in the system. Nominally, at least two pumps are needed: one involved in moving cells into the electroporation buffer, and one that later returns cells to a culture buffer. In order to protect against flow rate differentials between devices, fluidic capacitors or reservoirs can be placed between devices that act as ballast. In this case, each microfluidic device has its own set of pumps to control flow rate. These flow rates and specifically the flow rates of the cells are monitored with the various OCT system 100 and the information is provided to the controller 44 enabling feedback control of the pumps 30, 32, 34, 36.

In system 10, cells to be electroporated are withdrawn from incubator 12 using pump 30, which can be a syringe pump capable of controlling fluid flow. System 10 also includes reservoir 22 and pump 32, e.g., a syringe pump, for supplying electroporation buffer to electroporation arrangement 16. High conductivity fluid for the sheath flow can be added from reservoir 24 by means of pump 34, e.g., a syringe pump. Cell medium is supplied to buffer exchange module 18 from reservoir 26, using pump 36, e.g., a syringe pump.

System 10 provides various options regarding the reservoir that houses the cargo to be incorporated into the cells (or into other types of membrane bound structures. In one example that uses primary human T cells, mRNA in electroporation buffer can be introduced from reservoir 22 into media exchange device 14, which transfers cells into the mRNA-laden electroporation media before it flows into electroporation device 16. Other arrangements supply a cargo such as plasmid DNA, single-stranded linear DNA, double-stranded linear DNA, linearized plasmid DNA, single-stranded donor oligonucleotides, ribonucleoproteins (e.g., Cas9 protein complexed with guide RNA), proteins, or small molecules from reservoir 22. Cells can also be manually suspended in electroporation media laden with cargo, introduced into incubator 12, and then flowed directly into flow electroporation device 16.

Silastic or other suitable tubing can be employed for some or all connections providing fluid communication between the various modules (components).

Controller 44 can include one or more computers, hardware, software, sensors, interfaces, etc. for controlling the operation of system 10 or components thereof, to reach partial or complete automation. In many embodiments, controller 44 controls the electrical parameters applied for electroporation and/or the acoustic frequencies employed in buffer exchange device 14 and, optionally, in buffer exchange device 18. Controller 44 can monitor or control incubator parameters, the operation of one or more of pumps 30, 32, 34 and/or 36, the flow and parameters of central and/or sheath streams described above and so forth based on the information from the monitoring OCT systems 100.

Various embodiments that can be included in system 10 and/or its operation are further described below.

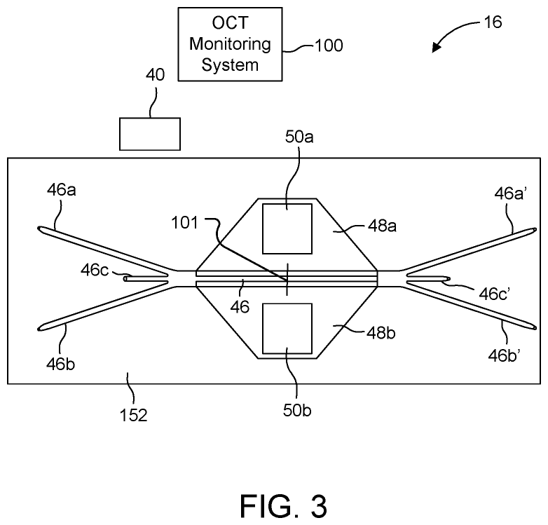

FIG. 3 is a top view of an exemplary electroporation assembly 16 that can support a sheath flow configuration such as described above. The arrangement includes microfluidic center channel 46 having trifurcating inlets (elements 46a, 46b and 46c) and trifurcating outlets (elements 46a′, 46b′ and 46c′).

Microfluidic channel 46 can be fabricated in a substrate 52 such as hard plastic (which, for many materials, renders the device disposable). Examples include but are not limited to cyclic olefin copolymer (COC) thermoplastic, a polyimide film, such as Kapton®, polystyrene, PEI (polyetherimide), e.g., Ultem®, or a combination of various polymers. Other materials such as glass, quartz, silicon, suitable ceramics, and so forth also can be employed.

The channel dimensions can range from 500 micrometer (μm) to 3 millimeter (mm) in width, 1 centimeter (cm) to 5 cm in length, and 125 μm to 500 μm in height. A pair of coplanar rhomboid-shaped electrodes (48a and 48b) are patterned onto the polymer layer beneath the floor of the microchannel with square wire bond or solder pad areas defined by cutouts in the polymer layers that expose the electrodes for external access. A masking layer is placed between the electrode layer and the microfluidic channel 46, with cutouts that define the portion of electrode that is exposed to fluid in the microchannel. Typically, the electrodes are formed from an electrochemically stable material, such as platinum metal (Pt). The portion of the electrodes that are exposed to the fluid in the channel have dimensions of 100-250 μm in width and 8-45 mm in length and interface to the electrical function generator 40 via connection to the square soldering pads (elements 50a and 50b in FIG. 3).

In an arrangement such as that of FIG. 3, the relative flow rates of the center vs. side streams can be tuned based on the monitoring by the OCT system 100 that monitors the cells in a cross section across the channel of the electroporation assembly 16, see line 101. In one example, the images provided by the OCT system 100 are provided to the controller 44 that controls the pumps to control the relative flow, which is adjusted so that the electrodes only make contact with the side streams. The total flow rate can range from 375 μL/min to 6 ml/min. In specific examples, the flow ratio for the side streams vs. the center stream is typically in the range of 1:0.5 to 1:1 (single side:center). When the conductivity of the solution comprising the center stream is much lower than the conductivities of the solutions comprising the side streams (e.g., 10× or more), the center stream dominates the electrical resistance of the circuit, such that, when voltage is applied to the electrodes, most of the voltage is dropped across the center stream.

The voltage (from the electrical function generator 40 in FIG. 2) is applied across the square soldering pads 50a and 50b and may take the form of sinusoids with periods ranging from 10 nanoseconds (ns) to 10 milliseconds (ms), or pulse trains with pulse widths ranging from 10 ns to 10 ms. The magnitude of the applied voltage can vary, so as to generate an electric field across the center stream that ranges from about 2-1000 kV/m, and pulse widths ranging from 10 ns to 10 ms. The frequency of the pulse train can be varied as well, and ranges, for example, from one pulse per cell residence time, to 10 pulses per residence time or more.

An arrangement such as that in FIG. 3 can support the use of different buffers. In specific embodiments, the sheath side streams are characterized by high electrical conductivity (a), e.g., in the range of from about 1 to about 2 Siemens per meter (S/m), while the central sheath stream has a low a, e.g., within a range of from 10 to 1000 micro Siemens per centimeter (μS/cm). This approach is compatible with the use of buffers suitable for cell culture and/or buffers suitable for electroporation.

Thus, in one implementation, the cells, in their preferred buffer, are provided via the center sheath stream 46c. The two side streams 46a and 46b are supplied from the high conductivity media reservoir 24 by pump 34 in FIG. 2. It is common for such a cell preferred buffer to have a high a, e.g., in the range of from about 10,000 to about 20,000 (μS/cm).

Low σ electroporation buffer flows in central stream 46c and is supplied from electroporation reservoir 22 by pump 32 in FIG. 2.

Prior to entering the electroporation module 16 (FIG. 3), cells coming from incubator 12 are flowed into the side stream port 110 of an acoustic media exchange module of FIG. 4A where they are driven or pushed, e.g., acoustically, from the high conductivity side sheath streams to the center stream, which contains a low a electroporation buffer and cargo. As a result, the cells become suspended in the central, electroporation buffer (which is then delivered to the center stream port 46c of module 16). The acoustic energy to drive the cells from one buffer to another is supplied from the acoustic function generator 42 to an acoustic transducer 154 attached to the channel substrate 152 as monitored by the OCT system 100.

After the electroporation operation (conducted in electroporation assembly 16 in FIGS. 2 and 3), cargo-containing product cells can remain suspended in the central stream and can be collected from outlet 46c′. Fluid obtained from outlets 46a′ and 46b′ is handled as waste or recycled. In other embodiments, a second buffer exchange (see buffer exchanger 18 in FIG. 1) can be performed to move the cargo-containing product cells from the low a electroporation buffer in the central stream to the high σ fluid in the side streams. In this configuration, cargo-containing product cells can be collected from outlets 46a′ and 46b′. Fluid from outlet 46c′ is handled as waste or directed to a collection arrangement for reuse.

While this invention has been particularly shown and described with references to preferred embodiments thereof, it will be understood by those skilled in the art that various changes in form and details may be made therein without departing from the scope of the invention encompassed by the appended claims.

What is claimed is:

1. A biological system comprising:
devices for modifying cells; and
a line-field swept source OCT system for monitoring the cells.

2. The system as claimed in claim 1, further comprising a controller for using information from the line-field swept source OCT to control the devices.

3. The system as claimed in claim 1, wherein the devices include pumps feedback controlled by the controller.

4. The system as claimed in claim 1, wherein the devices include at least one incubator and a line-field swept source OCT system for monitoring cells in the incubator.

5. The system as claimed in claim 1, wherein the devices include at least one buffer exchange device and a line-field swept source OCT system for monitoring cells in the buffer exchange device.

6. The system as claimed in claim 1, wherein the devices include flow electroporation device and a line-field swept source OCT system for monitoring cells in the flow electroporation device.

7. The system as claimed in claim 1, wherein the line-field swept source OCT system includes:
a base;
a swept laser on the base;
a beamsplitter on the base for dividing the beam from the swept laser between a reference arm and a sample arm; and
a line-field sensor for detecting light from the reference arm and the sample arm.

8. The system of claim 7, further comprising a bracket for mounting the line-field sensor to the base.

9. The system of claim 7, further comprising a line generating lens for forming a line from the beam from the laser.

10. The system of claim 9, wherein the line generating lens forms a less Gaussian and more of a flat-top power distribution of the light from the laser.

11. The system of claim 9, wherein the line generating lens is a Powell lens.

12. The system of claim 7, further comprising a translation stage for changing a length of the reference arm.

13. A control method for a biological system comprising:
modifying cells in the biological system;
monitoring the cells in the biological system with a line-field swept source OCT system; and
controlling the biological system based on information from the line-field swept source OCT system.

14. The method as claimed in claim 13, further controlling pumps based on information from the line-field swept source OCT system.

15. The method as claimed in claim 13, further controlling at least one incubator based on information from the line-field swept source OCT system.

16. The method as claimed in claim 13, further comprising at least one buffer exchange device based on information from the line-field swept source OCT system.

17. The method as claimed in claim 13, further comprising controlling a flow electroporation device based on information from the line-field swept source OCT system.
